(19) United States
(12) Patent Application Publication
(10) Pub. No.: US 2009/0104266 A1
(43) Pub. Date: Apr. 23, 2009

(54) 3-(2-DIMETHYLAMINOMETHYLCY CLOHEXYL)PHENOL RETARD FORMULATION

(76) Inventors: Tobias Jung, Efringen-Kirchen (DE); Johannes Bartholomaus, Aachen (DE)

Correspondence Address: CROWELL & MORING LLP;INTELLECTUAL PROPERTY GROUP, P.O. BOX 14300, WASHINGTON, DC, 20044-4300, US

(21) Appl. No.: 12/066,927

(22) PCT Filed: Sep. 15, 2005
(86) PCT No.: PCT/EP2006/008988

§ 371 Date: Sep. 15, 2008

(30) Foreign Application Priority Data
Sep. 15, 2005 (DE) 10 2005 044 212.9

(51) Int. Cl. A61K 9/10; C07C 215/64; A61P 25/00; A61K 31/138

(52) U.S. Cl. 424/488; 564/443; 514/646; 424/484

(57) ABSTRACT

The invention relates to a dosage form for controlled release of the active ingredient 3-(2-dimethylaminomethylcyclohexyl)phenol, preferably (1R,2R)-3-(2-dimethylamino-methylcyclohexyl)phenol, or one of the pharmaceutically acceptable salts thereof, which
(i) in vivo achieves the peak plasma level of the active ingredient after 2 to 10 h,
and/or
(ii) in vitro, measured in accordance with the European Pharmacopoeia with a paddle stirrer apparatus in buffer at a pH value of 6.8 (preferably 900 ml), a temperature of 37° C. and 75 rpm releases
   after 0.5 hours 3.0 to 37 wt. %,
   after 1 hour 5.0 to 56 wt. %,
   after 2 hours 10 to 77 wt. %,
   after 3 hours 15 to 88 wt. %,
   after 6 hours at least 30 wt. %,
   after 12 hours at least 50 wt. %,
   after 18 hours at least 70 wt. % and
   after 24 hours at least 80 wt. %
of the active ingredient originally contained in the dosage form.

The invention relates to a pharmaceutical dosage form with controlled release of 3-(2-dimethylaminomethylcyclohexyl)phenol, preferably of the (1R,2R) stereoisomer, or one of the pharmaceutically acceptable salts thereof.

3-(2-Dimethylaminomethylcyclohexyl)phenol is known from the prior art. It is an orally administrable, analgesically active pharmaceutical substance (cf. for example DE-A 195 25 137, WO 02/43712 and WO 02/67916).

Due to the two chiral centres, 3-(2-dimethylaminomethylcyclohexyl)phenol occurs in the form of four stereoisomers (two enantiomeric pairs), namely as (1R,2R)-, (1S,2S)-, (1R,2S)- and (1S,2R)-3-(2-dimethylaminomethylcyclohexyl)phenol. These four stereoisomers are of the following structure:

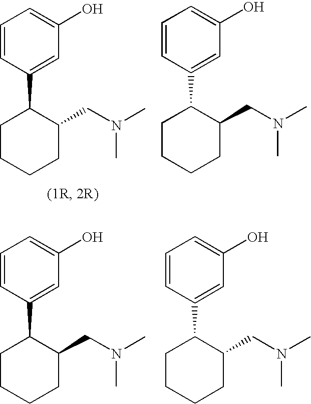

Conventional dosage forms for oral administration of 3-(2-dimethylaminomethylcyclohexyl)phenol lead to rapid release of the entire active ingredient dose in the gastrointestinal tract, resulting in rapid onset of the analgesic action. Consequently, treating severe chronic pain with 3-(2-dimethylaminomethylcyclohexyl)phenol has hitherto meant administering the medicament at relatively short time intervals, for example four to six times daily, in order to achieve an adequate active ingredient concentration in the patient's plasma over a 24 h period.

However, the necessity of administering frequent doses often results in errors in taking and in undesirable fluctuations in plasma concentration, which have a negative impact on compliance and therapeutic benefit, in particular in the treatment of chronic pain. It is furthermore known that, with conventional dosage forms, oral administration of 3-(2-dimethylaminomethylcyclohexyl)phenol may result in side-effects, in particular nausea and vomiting.

It is known to provide dosage forms with controlled-release of the active ingredients contained therein in order to ensure continuous release of the active ingredient over an extended period.

Controlled-release formulations for a large number of active ingredients are known in the prior art. Controlled release is conventionally achieved by suitable coatings and/or by embedding the active ingredient in a matrix which controls release.

In the case of coated controlled-release formulations, an active ingredient-containing core is provided with a coating of hydrophilic and/or hydrophobic polymers which delays release of the active ingredient. In the case of matrix controlled-release formulations, the active ingredient is embedded in a polymer matrix which controls release of the active ingredient.

However, if a specific release profile is to be achieved for an active ingredient, it is not straightforwardly possible, starting from a known prior art composition having the desired release profile, simply to swap the active ingredient contained therein. Instead, the individual physical and chemical properties of the particular active ingredient must be taken into account for each individual case. Numerous individual properties of an active ingredient may accordingly have a considerable influence on its release profile. The specific release behaviour of an active ingredient may be determined, for example, by the dose to be administered, particle size, particle shape, hardness, hygroscopicity, solubility, dependency of solubility on pH value, hydrophilicity/lipophilicity, acidity/basicity, etc.

The object of the present invention is to provide a pharmaceutical formulation of 3-(2-dimethylaminomethylcyclohexyl)phenol, preferably the (1R,2R) stereoisomer thereof or one of the pharmaceutically acceptable salts thereof, which has advantages over prior art formulations.

The dosage form should accordingly provide pharmacologically active plasma concentrations of the active ingredient 3-(2-dimethylaminomethylcyclohexyl)phenol over an extended period, preferably for at least 12 h, (controlled release) and, in so doing, be characterised by the smallest possible range of side-effects, in particular with regard to nausea and/or vomiting. Pharmacokinetic behaviour should furthermore differ to a great extent from the pharmacokinetic behaviour of a comparison formulation without controlled release (active ingredient solution, succus, immediate release).

This object is achieved by the subject matter of the claims.

It has surprisingly been found that it is possible to produce a dosage form of the active ingredient 3-(2-dimethylaminomethylcyclohexyl)phenol or one of the pharmaceutically acceptable salts thereof which releases the active ingredient in controlled manner and, in so doing, has advantages over prior art dosage forms.

The invention relates to a dosage form for controlled release of the active ingredient 3-(2-dimethylaminomethylcyclohexyl)phenol, preferably (1R,2R)-3-(2-dimethylaminomethylcyclohexyl)phenol, or one of the pharmaceutically acceptable salts thereof, which
(i) in vivo achieves the peak plasma level of the active ingredient after 2 to 10 h,
and/or
(ii) in vitro, measured in accordance with the European Pharmacopoeia with a paddle stirrer apparatus in buffer at a pH value of 6.8 (preferably 900 ml), a temperature of 37° C. and 75 rpm releases
   after 0.5 hours 3.0 to 37 wt. %,
   after 1 hour 5.0 to 56 wt. %,
   after 2 hours 10 to 77 wt. %,
   after 3 hours 15 to 88 wt. %,
   after 6 hours at least 30 wt. %,
   after 12 hours at least 50 wt. %,
   after 18 hours at least 70 wt. % and
   after 24 hours at least 80 wt. %
of the active ingredient originally contained in the dosage form.

The following table shows particularly preferred release profiles of the dosage form according to the invention (no. 1 to no. 8):

| after | 1 | 2 | 3 | 4 | 5 | 6 | 7 | 8 |
|---|---|---|---|---|---|---|---|---|
| [h] | wt. % | wt. % | wt. % | wt. % | wt. % | wt. % | wt. % | wt. % |
| 0.5 | 5.0-34 | 6.0-33 | 7.0-32 | 9.0-31 | 11-30 | 13-30 | 15-29 | 17-28 |
| 1 | 12-53 | 15-52 | 18-50 | 20-48 | 22-46 | 24-44 | 27-42 | 30-40 |
| 2 | 25-74 | 27-71 | 29-68 | 31-65 | 33-62 | 36-60 | 39-58 | 42-56 |
| 3 | 33-85 | 36-82 | 39-79 | 42-76 | 45-73 | 48-71 | 50-69 | 52-67 |
| 4 | 41-92 | 44-89 | 47-86 | 50-83 | 53-81 | 55-79 | 58-77 | 60-75 |
| 6 | 52-98 | 55-97 | 58-96 | 60-94 | 63-92 | 66-90 | 69-88 | 72-86 |
| 8 | >62 | >65 | >68 | 71-99 | 74-98 | 76-98 | 78-97 | 80-97 |
| 12 | >70 | >73 | >76 | >79 | >82 | >84 | >86 | >88 |

The dosage form according to the invention releases the active ingredient 3-(2-dimethylaminomethylcyclohexyl)phenol, preferably after oral administration, in delayed manner and is thus suitable for administration at an interval of at least 12 h. The dosage form according to the invention accordingly permits pain therapy which requires the administration of the active ingredient 3-(2-dimethylaminomethylcyclohexyl)phenol only once daily, for example at intervals of 24 h, or twice daily, preferably at intervals of 12 hours, in order to ensure an adequate plasma concentration of the active ingredient.

It has here surprisingly been found that, in comparison with conventional dosage forms intended for oral administration of 3-(2-dimethylaminomethylcyclohexyl)phenol, it is possible to achieve a significant reduction in side-effects, in particular nausea and/or vomiting. This has the advantage of increasing the therapeutic range (ratio of the therapeutic dose to the toxic active ingredient dose) of 3-(2-dimethylaminomethylcyclohexyl)phenol, as a result of which it is possible inter alia to increase the active ingredient dose and thus also therapeutic efficacy.

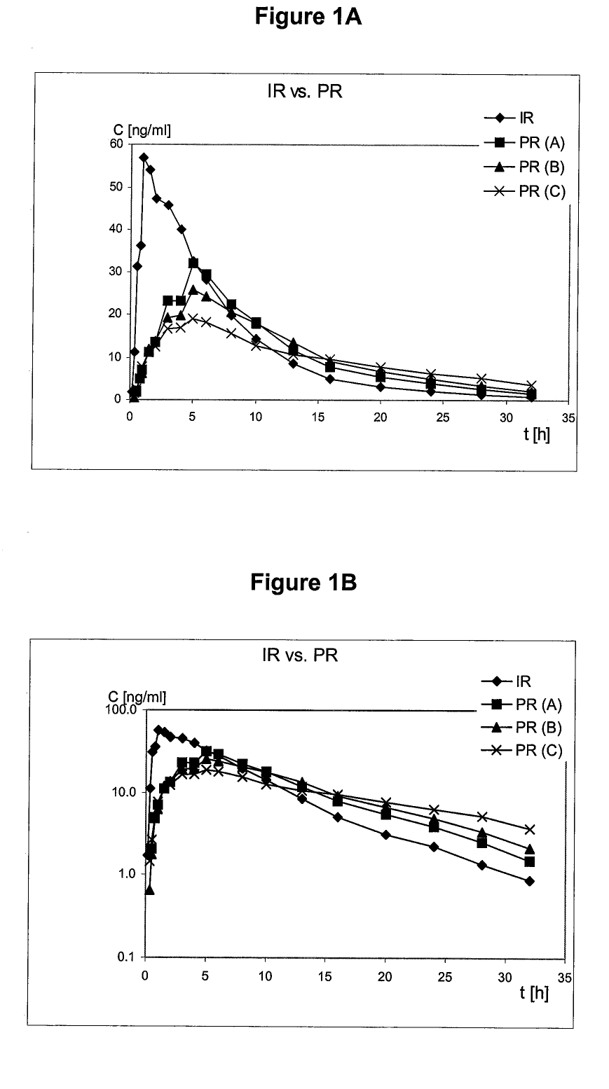

FIG. 1 shows average plasma concentrations of the active ingredient 3-(2-dimethylaminomethylcyclohexyl)phenol after oral administration of dosage forms according to the invention with controlled release PR (A), PR (B) and PR (C) in comparison with a conventional dosage form (comparison formulation without controlled release, active ingredient solution, succus, IR). In FIG. 1A, the y-axis is linear, while in FIG. 1B it is logarithmic.

In a conventional dosage form without controlled release (immediate release IR, active ingredient solution, succus) it may usually be assumed that, very shortly after administration, the course of the plasma concentration/time curve is substantially solely determined by the kinetics of metabolisation and excretion of the active ingredient from the body. Such kinetics vary from active ingredient to active ingredient (substance constant) but are virtually independent of the formulation of the dosage form.

In contrast, in a conventional dosage form with controlled release, it may usually be assumed that the formulation of the dosage form has an influence on the course of the plasma concentration/time curve for as long as active ingredient is still being released from the dosage form. Over this interval of time, the kinetics of resupply of the active ingredient from the dosage form and the kinetics of metabolisation and excretion of the active ingredient which has already been released are superimposed on one another. However, from a certain point in time, release of the active ingredient from the dosage form has progressed so far that almost no further active ingredient is supplied. The kinetics of resupply of the active ingredient from the dosage form then has at most only a slight influence on the course of the plasma concentration/time curve. Instead, in this phase the course of the plasma concentration/time curve is substantially still only dominated by the kinetics of metabolisation and excretion of the active ingredient from the body; accordingly, over this period of time, the course of the plasma concentration/time curve becomes similar to the course observed in dosage forms without controlled release.

However, as is clear from FIG. 1, in the dosage forms according to the invention (PR (A), PR (B) and PR (C)), sometimes even many hours after administration, the course of the plasma concentration/time curve does not closely follow the course of the plasma concentration/time curve of a comparison formulation without controlled release (active ingredient solution, succus, IR). As is in particular clear from FIG. 1B, even many hours after administration, the gradients in the terminal part of the course of the plasma concentration/time curve in the dosage forms according to the invention with controlled release (PR (A), PR (B) and PR (C)) are distinctly different from the gradient of the comparison formulation without controlled release (the terminal run-outs of the curves do not have a parallel course, but are instead a bundle of curves).

In the terminal elimination phase in the plasma concentration/time diagram with logarithmic linear regression, the negative gradient of the regression lines is defined as the rate constant λ_{z}. The surprising behaviour of the dosage form with controlled release according to the invention in comparison with a conventional dosage form without controlled release may therefore be identified by λ_{z} or the half-life derived therefrom.

This surprising behaviour of the dosage form according to the invention has the advantage that, in vivo, the dosage form still has a controlled-release action on the plasma concentration of the active ingredient at times at which, on the basis of the in vitro release rate data, no further significant controlled-release action would be anticipated. The in vivo controlled-release action (measured plasma concentration) is accordingly enhanced relative to the in vitro controlled-release action (measured release values); it has a superproportional action. The expected duration of action is accordingly extended and compatibility is further improved in comparison with the situation to be anticipated without this in vivo effect.

For the purposes of the description, “controlled” release of the active ingredient may, within the bounds of the invention, be delayed release (extended release), repeat action release, prolonged release or sustained release. Immediate active ingredient release (immediate release), such as is achieved for example with the assistance of an active ingredient solution, should not be understood for the purposes of the description to mean controlled release of the active ingredient.

In a preferred embodiment of the dosage form according to the invention, the dosage form comprises a polymer matrix which releases preferably at least a proportion of the entirety of the active ingredient contained in the dosage form in delayed manner (matrix controlled-release formulation). To this end, at least this proportion of the active ingredient, preferably (1R,2R)-3-(2-dimethylaminomethylcyclohexyl)phenol or one of the pharmaceutically acceptable salts thereof, is present embedded in the polymer matrix.

In another preferred embodiment of the dosage form according to the invention, the dosage form comprises a film coating which releases preferably at least a proportion of the entirety of the active ingredient contained in the dosage form in delayed manner (coated controlled-release formulation).

It is also possible to combine a matrix controlled-release formulation with a coated controlled-release formulation. However, according to the invention, release of the active ingredient is preferably substantially exclusively controlled by a polymer matrix.

In a preferred embodiment, the dosage form according to the invention comprises a polymer matrix which preferably comprises one or more hydrophilic or hydrophobic, pharmaceutically acceptable polymers, for example cellulose ethers, cellulose esters, polyethylene glycols (PEG), gums, (meth)acrylates, protein-derived material, fats, waxes, fatty alcohols and/or fatty acid esters. When hydrophilic polymers are used as matrix former, it is preferred for the proportion by weight of the polymer matrix to amount to 5.0 to 85 wt. %, preferably 20 to 60 wt. %, relative to the total weight of the dosage form according to the invention.

The dosage form according to the invention preferably comprises a polymer matrix, in which at least a proportion of the active ingredient, preferably (1R,2R)-3-(2-dimethylaminomethylcyclohexyl)phenol or one of the pharmaceutically acceptable salts thereof, is embedded, wherein the polymer matrix is based on a cellulose ether and/or cellulose ester, which at a concentration of 2.0 wt. % in an aqueous solution at 20° C. has a viscosity, preferably determined by capillary viscosimetry according to the European Pharmacopoeia, in the range from 3,000 to 150,000 mPa·s, preferably from 5,000 to 145,000 mPa·s, more preferably from 10,000 to 140,000 mPa·s, still more preferably from 25,000 to 135,000 mPa·s, most preferably from 50,000 to 130,000 mPa·s and in particular from 80,000 to 120,000 mPa·s.

In a preferred embodiment of the dosage form according to the invention, the polymer matrix comprises at least one cellulose ether and/or cellulose ester selected from the group consisting of methylcellulose (MC), ethylcellulose (EC), hydroxyethylcellulose (HEC), hydroxypropylcellulose (HPC), carboxymethylcellulose (CMC) and hydroxypropylmethylcellulose (HPMC). Most preferred are HPMCs with a viscosity of approx. 100,000 mPa·s, measured in a 2 wt. % aqueous solution at 20° C. Alternatively or additionally, the matrix may also have a content of polyethylene glycols (PEG) of 0 to 60 wt. %.

The proportion by weight of the polymer matrix is preferably in the range from 5.0 to 85 wt. %, more preferably from 10 to 50 wt. % and most preferably from 25 to 45 wt. %, relative to the total weight of the dosage form. When determining the proportion by weight, account is preferably taken only of those polymers of the dosage form according to the invention which form a matrix in which at least a proportion of the active ingredient is embedded. Conventional polymeric auxiliaries, such as for example microcrystalline cellulose, in contrast are not included in the determination if they play virtually no part in matrix formation.

In a preferred embodiment of the dosage form according to the invention, the proportion by weight of the active ingredient, preferably (1R,2R)-3-(2-dimethylaminomethylcyclohexyl)phenol or one of the pharmaceutically acceptable salts thereof, is in the range from 0.5 to 85 wt. %, more preferably from 5.0 to 50 wt. % and most preferably from 15 to 35 wt. %, relative to the total weight of the dosage form.

In the dosage forms according to the invention, the active ingredient content is preferably between 0.5 and 85 wt. % and the content of the polymer matrix is between 8.0 and 40 wt. %. Particularly preferred dosage forms are those having an active ingredient content of between 3.0 and 70 wt. %, in particular between 8.0 and 66 wt. %, and a polymer matrix content of between 10 and 35 wt. %, in particular between 10 and 30 wt. %, relative to the total weight of the pharmaceutical composition.

The relative weight ratio of the polymer matrix to the active ingredient, preferably to (1R,2R)-3-(2-dimethylaminomethylcyclohexyl)phenol, or to one of the pharmaceutically acceptable salts thereof, is preferably in the range from 3:1 to 1:10, more preferably from 2.5:1 to 1:8, still more preferably from 2.2:1 to 1:5 and most preferably from 2:1 to 1:2.

In a particularly preferred embodiment of the dosage form according to the invention, the polymer matrix comprises hydroxypropylmethylcellulose which at a concentration of 2.0 wt. % in an aqueous solution at 20° C. has a viscosity, preferably determined by capillary viscosimetry according to the European Pharmacopoeia, in the range from 50,000 to 130,000 mPa·s, wherein simultaneously the proportion by weight of hydroxypropylmethylcellulose is in the range from 15 to 35 wt. %, relative to the total weight of the dosage form.

The pharmaceutical dosage forms according to the invention may furthermore contain usual pharmaceutical auxiliary materials as further constituents, such as for example
fillers, for example lactose, microcrystalline cellulose (MCC) or calcium hydrogenphosphate, and/or
slip, lubricant and flow-control agents, for example talcum, magnesium stearate, stearic acid and/or highly disperse silicon dioxide,
   wherein the total content thereof in the tablet is preferably between 0 and 80 wt. %, more preferably between 5.0 and 65 wt. %.

Such auxiliary materials are known to a person skilled in the art. In this connection, reference may for example be made to the full content of H. P. Fiedler, Lexikon der Hilfsstoffe für Pharmazie, Kosmetik und angrenzende technische Gebiete, Editio Cantor Aulendorff, 2002.

The dosage form according to the invention preferably contains at least one filler, wherein the relative weight ratio of the filler or the total of all fillers to the polymer matrix is less than 6:1, more preferably in the range from 5:1 to 1:2, still more preferably from 4:1 to 1:1.5 and most preferably from 3:1 to 1:1.

In a preferred embodiment of the dosage form according to the invention it contains a filler selected from the group consisting of
fillers soluble in an aqueous medium, for example lactose,
non-swelling fillers insoluble in an aqueous medium, for example calcium hydrogenphosphate; and
swelling fillers insoluble in an aqueous medium, for example microcrystalline cellulose.

The filler is preferably selected from the group consisting of microcrystalline cellulose, calcium hydrogenphosphate and lactose.

The release profiles of the active ingredient, preferably (1R,2R)-3-(2-dimethylaminomethylcyclohexyl)phenol or one of the pharmaceutically acceptable salts thereof, from the dosage form according to the invention are preferably independent of the pH value as may prevail physiologically during passage through the gastrointestinal tract. The release profiles at a pH value of the surroundings of 1.2 and 6.8 are preferably both substantially identical to one another and also in comparison with release over pH value time profile from pH 1.2 through pH 2.3 and through pH 6.8 up to pH 7.2.

The dosage form according to the invention contains 3-(2-dimethylaminomethylcyclohexyl)phenol or one of the pharmaceutically acceptable salts thereof as active ingredient. The active ingredient may here be present as a mixture of two or more of the stereoisomers (enantiomers and/or diastereomers) thereof. 3-(2-Dimethylaminomethylcyclohexyl)phenol may be present in the dosage form according to the invention not only as a mixture of all four diastereomers in any desired mixing ratio, but also as a mixture of two or three of the four stereoisomers or in stereoisomerically pure form. In a preferred embodiment, the active ingredient is present as a racemic compound of the (1R,2R)/(1S,2S) enantiomeric pair, wherein preferably neither the (1R,2S), nor the (1S,2R) diastereomer is present or the proportion by weight thereof is less than 2.0 wt. %, relative to the total weight of the active ingredient.

Pharmaceutically acceptable salts of the active ingredient for the purposes of the present invention are such salts of the active ingredient, which, when used pharmaceutically, are physiologically compatible, in particular for use in mammals and/or humans. Such pharmaceutically acceptable salts may, for example, be formed with inorganic or organic acids. Examples of salts of inorganic acids which may be mentioned are: hydrochlorides, hydrobromides, sulfates, phosphates, hydrogenphosphates and dihydrogenphosphates. Examples of salts of organic acids which may be mentioned are: formates, acetates, propionates, fumarates, glutarates, pyruvates, malates, tartrates, benzoates, citrates, ascorbates, maleates, etc.

In a particularly preferred embodiment, the dosage form according to the invention contains the stereoisomer (1R,2R)-3-(2-dimethylaminomethylcyclohexyl)phenol or one of the pharmaceutically acceptable salts thereof, wherein the enantiomeric excess thereof preferably amounts to at least 90% ee, more preferably at least 95% ee, still more preferably at least 97% ee and in particular at least 98% ee.

The active ingredient 3-(2-dimethylaminomethylcyclohexyl)phenol may be present not only as such, i.e. as the free base, but also in the form of a pharmaceutically acceptable salt, for example as the hydrochloride. Production of the hydrochlorides is known, for example, from DE-A 195 25 137. Prior art methods are known for converting the hydrochloride into the free base or into another pharmaceutically acceptable salt. Methods for separating the enantiomers or diastereomers are also sufficiently well known in the prior art. The diastereomers may, for example, be separated by HPLC and the enantiomers by HPLC on chiral stationary phases.

In addition to the active ingredient 3-(2-dimethylaminomethylcyclohexyl)phenol or one of the pharmaceutically acceptable salts thereof, the dosage form according to the invention may contain further pharmaceutically active substances. Preferably, however, the dosage form according to the invention contains only 3-(2-dimethylaminomethylcyclohexyl)phenol, preferably (1R,2R)-3-(2-dimethylaminomethylcyclohexyl)phenol or one of the pharmaceutically acceptable salts thereof, and otherwise no further pharmaceutically active substances.

Preferred embodiments of the dosage form according to the invention (no. 1 to no. 5) comprise the following constituents in the following quantities (percentages are in each case relative to the total weight of the dosage form):

| | wt. % | | | | |
|---|---|---|---|---|---|
| Constituent | 1 | 2 | 3 | 4 | 5 |
| Active ingredient, preferably (1R,2R)-3-(2- | 1.0-50 | 2.5-45 | 5.0-41 | 15-35 | 20-30 |
| dimethylaminomethylcyclohexyl)phenol | | | | | |
| Cellulose ether or cellulose ester, preferably | 5.0-75 | 7.5-60 | 9.0-50 | 15-45 | 25-40 |
| MC, EC, HEC, HPC, CMC or HPMC | | | | | |
| Filler, preferably microcrystalline cellulose, | 10-90 | 20-75 | 30-66 | 35-60 | 40-55 |
| calcium hydrogenphosphate or lactose | | | | | |
| Flow-control agent, preferably highly disperse | 0-5.0 | 0-2.5 | 0-1.0 | 0.1-1.0 | 0.2-0.8 |
| silicon dioxide | | | | | |
| Slip agents, preferably magnesium stearate | 0-5.0 | 0-2.5 | 0-1.0 | 0.1-1.0 | 0.2-0.8 |

Further constituents of the dosage form according to the invention may be optionally digestible long-chain (i.e. with 8 to 50 C atoms, preferably 12 to 40 C atoms) unsubstituted or substituted hydrocarbons, such as for example fatty alcohols, fatty acid glyceryl esters, mineral and vegetable oils, as well as waxes, wherein hydrocarbons with a melting point of between 25° and 90° C. are preferred. In particular, fatty alcohols are preferred, most particularly lauryl alcohol, myristyl alcohol, stearyl alcohol, cetyl alcohol and cetyl stearyl alcohol. The content thereof in the dosage form is preferably 0 to 20 wt. %.

The dosage form according to the invention is characterised by advantageous pharmacokinetic parameters.

For the purposes of the description, the pharmacokinetic parameters, which may be determined from the blood plasma concentrations of 3-(2-dimethylaminomethylcyclohexyl)phenol, are defined as follows:

| | |
|---|---|
| τ | Dose range |
| Cₘₐₓ | maximum measured plasma concentration of the active |
| | ingredient after single administration (≡average peak plasma level) |
| Cₘᵢₙ | minimum measured plasma concentration of the active |
| | ingredient after single administration |
| Cₐᵥ | average plasma concentration of the active ingredient after |
| | $single administration \text{:} {C}_{av} = \frac{{AUC}_{τ}}{τ}$ |
| tₘₐₓ | interval of time from administration of the active ingredient |
| | until Cₘₐₓ is reached |
| t_{lag}, t(0) | interval of time from administration until the first detectable |
| | plasma concentration of the active ingredient (lag time) |
| AUC_{τ} | total area of the plasma concentration/time curve during τ |
| AUC₀₋ₜ | total area of the plasma concentration/time curve from |
| | administration until the final measured value |
| AUC_{t-∞} | subarea of the plasma concentration/time curve from the final |
| | measured value extrapolated to infinity |
| AUC | total area of the plasma concentration/time curve including the |
| | subarea from the final measured value extrapolated to infinity |
| AUC %ₑₓₜᵣ | subarea of the plasma concentration/time curve from the final |
| | measured value extrapolated to infinity in percent |
| λ_{z} | rate constant of the terminal elimination phase, defined as the |
| | negative gradient of the regression lines with logarithmic |
| | linear regression |
| t_{1/2, z} | $half \text{-} life during the terminal elimination phase \text{:} {t}_{1 / 2 , z} = \frac{ln ( 2 )}{{λ}_{z}}$ |
| HVD | half value duration, defined as the time interval over which |
| | plasma concentration is greater than 50% of Cₘₐₓ |
| MRT | mean residence time, defined as the ratio of AUMC (area |
| | under the “first moment curve”) and AUC (cf. M. Gibaldi, |
| | D. Perrier, J Pharm. Sci. 1982, 71(4), 474-5) |
| CL/f | $total clearance after oral administration \text{:} CL / f = \frac{Dose}{AUC}$ |
| V_{z}/f | apparent distribution volume during the terminal disposition |
| | $phase after oral administration \text{:} {V}_{z} / f = \frac{Dose}{AUC \cdot {λ}_{z}}$ |
| PTF | peak to trough fluctuation over an administration period: |
| | $PTF \% = 100 \frac{{C}_{max} - {C}_{min}}{{C}_{av}}$ |

The above parameters are in each case stated as mean values of the individual values for all investigated patients/test subjects.

A person skilled in the art knows how the pharmacokinetic parameters of the active ingredient 3-(2-dimethylaminomethylcyclohexyl)phenol may be calculated from the measured concentrations of the active ingredient in the blood plasma. In this connection, reference may be made, for example, to Willi Cawello (ed.) Parameters for Compartment-free Pharmacokinetics, Shaker Verlag Aachen (1999).

After preferably oral administration of the dosage form according to the invention, in vivo the average peak plasma level (Cₘₐₓ) is on average preferably reached after tₘₐₓ 2 to 10 h, more preferably after 3 to 8 h, still more preferably after 3.5 h to 6 h, most preferably after 4.0 to 5.5 h and in particular after 4.2 to 5.2 h.

The average value for MRT after preferably oral administration of the dosage form according to the invention in vivo is preferably more than 7.5 h, more preferably more than 8.0 h, still more preferably more than 9.0 h; it is most preferably in the range from 10.0 to 25.0 h and in particular in the range from 11.0 to 20.0 h.

The average value for HVD after preferably oral administration of the dosage form according to the invention in vivo is preferably more than 5.0 h, more preferably more than 6.0 h, still more preferably more than 7.0 h; it is most preferably in the range from 8.0 to 20.0 h and in particular in the range from 9.0 to 18.0 h.

The average value for λ_{z} at identical dose is preferably lower than in a comparison formulation without controlled release. The average value for λ_{z} after preferably oral administration of the dosage form according to the invention in vivo is preferably less than 0.125 h⁻¹, more preferably less than 0.122 h⁻¹, still more preferably less than 0.118 h⁻¹; it is most preferably in the range from 0.050 to 0.115 h⁻¹ and in particular in the range from 0.060 to 0.112 h⁻¹.

The average value for t_{1/2,z} at identical dose is preferably higher than in a comparison formulation without controlled release. The average value for t_{1/2,z} after preferably oral administration of the dosage form according to the invention in vivo is preferably more than 5.7 h, more preferably more than 6.0 h, still more preferably more than 6.2 h; it is most preferably in the range from 6.4 h to 20.0 h and in particular in the range from 6.6 h to 15.0 h.

A “comparison formulation without controlled release” is taken for the purposes of the description to mean an immediate release formulation of the active ingredient, for example a succus, for example an active ingredient solution or active ingredient dispersion with identical dosage. In a preferred embodiment, this is taken to mean an active ingredient solution, 1 ml of which contains the following constituents:
10.0 mg 3-(2-dimethylaminomethylcyclohexyl)phenol or an equivalent dose of one of the pharmaceutically acceptable salts thereof
7.0 mg sodium chloride, Ph.Eur., for parenteral use
0.5 mg sodium citrate.2H₂O, Ph.Eur., for parenteral use
985.5 mg water for injection, Ph.Eur.*

In another preferred embodiment, a “comparison formulation without controlled release” is taken to mean a capsule formulation comprising the auxiliary substances microcrystalline cellulose, low-substituted hydroxypropylcellulose, magnesium stearate and silicon dioxide, preferably of the following composition:
30 mg 3-(2-dimethylaminomethylcyclohexyl)phenol or an equivalent dose of one of the pharmaceutically acceptable salts thereof,
231 mg microcrystalline cellulose, PH102, Ph.Eur.,
72 mg low-substituted hydroxypropylcellulose, NF (L-HPC, grade LH 11),
18 mg magnesium stearate, Ph.Eur., and
9 mg highly disperse silicon dioxide, Ph.Eur.;
Total weight: 360 mg, preferably in a hard gelatine capsule, size 0el.

At dose D of the active ingredient, the average value for Cₘₐₓ/D after preferably oral administration of the dosage form according to the invention in vivo is preferably 7.0 10⁻⁵ l⁻¹≦Cₘₐₓ/D≦1.05 10⁻³ l⁻¹, more preferably 8.0 10⁻⁵ l⁻¹≦Cₘₐₓ/D≦1.0 10⁻³ l⁻¹, still more preferably 9.0 10⁻⁵ l⁻¹≦Cₘₐₓ/D≦9.0 10⁻⁴ l⁻¹, most preferably 1.0 10⁻⁴ l⁻¹≦Cₘₐₓ/D≦8.0 10⁻⁴ l⁻¹, and in particular 2.0 10⁻⁴ l⁻¹≦Cₘₐₓ/D≦7.0 10⁻⁴ l⁻¹.

The average value for Cₘₐₓ/AUC after preferably oral administration of the dosage form according to the invention in vivo is preferably between 0.150 and 0.010 h⁻¹, more preferably between 0.125 and 0.020 h⁻¹, still more preferably between 0.100 and 0.030 h⁻¹, most preferably between 0.095 and 0.040 h⁻¹ and in particular between 0.090 and 0.050 h⁻¹. The value for Cₘₐₓ/AUC may be regarded as a surrogate for absorption rate.

In the case of twice daily administration, preferably at intervals of 12 h, the average value for PTF is preferably <80%, more preferably ≦75%, still more preferably ≦70%, most preferably ≦65% and in particular ≦60%.

The dosage form according to the invention contains the active ingredient 3-(2-dimethylaminomethylcyclohexyl)phenol, preferably (1R,2R)-3-(2-dimethylaminomethylcyclohexyl)phenol, as such and/or as a pharmaceutically acceptable salt in a quantity of conventionally 2.5 to 800 mg, in particular 5 to 400 mg, very particularly preferably 10 to 250 mg (weight relative to 3-(2-dimethylaminomethylcyclohexyl)phenol as hydrochloride) per dosage unit, wherein the release behaviour of the dosage form according to the invention is virtually unaffected by the exact quantity of active ingredient, provided that the above-stated content limits are observed.

A preferred embodiment of the dosage form according to the invention is formulated for oral or rectal administration, preferably once or twice daily. If taken once or twice daily, a pharmaceutical dosage form according to the invention reliably achieves good therapeutic effectiveness in patients with chronic, severe pain.

The pharmaceutical dosage forms according to the invention may assume the form of both simple tablets and coated tablets, for example film tablets or sugar-coated tablets. The tablets are conventionally round or biconvex; oblong tablet shapes, which allow the tablet to be divided, are also possible. Granules, spheroids, pellets or microcapsules are also possible, which are packaged in sachets or capsules or may be compressed to form disintegrating tablets.

It is also possible, in addition to or as an alternative to the delayed release matrix in the pharmaceutical dosage form, to use a coating which controls release of the active ingredient. The active ingredient, which is preferably, but not necessarily, embedded in a polymer matrix, and optionally further pharmaceutical auxiliaries, such as for instance binders, fillers, slip, lubricant and flow-control agents, may be present in this case and be covered or coated with a material which controls and/or modulates delayed release of the active ingredient in an aqueous medium. Suitable coating materials are, for example, water-insoluble waxes and polymers, such as polymethacrylates (Eudragit or the like) or water-insoluble celluloses, in particular ethylcellulose. The coating material may optionally also contain water-soluble polymers, such as polyvinylpyrrolidone, water-soluble celluloses, such as hydroxypropylmethylcellulose or hydroxypropylcellulose, other water-soluble agents, such as Polysorbate 80, or hydrophilic pore formers, such as polyethylene glycol, lactose or mannitol.

One or more coating layers may be used for the coated dosage forms, preferably tablets. Known hydroxypropylmethylcelluloses with a low viscosity of approx. 1.0 to 100 mPa·s and a low molecular weight of <10,000 g mol⁻¹ (for example Pharmacoat 606 with a viscosity of 6.0 mPa·s in a 2.0 wt. % aqueous solution at 20° C.), which have virtually no or only a slight effect on the release profile of the medicament according to the invention, are suitable as coating materials.

Diffusion coatings known to a person skilled in the art, for example based on swellable, but water-insoluble poly(meth)acrylates, modulate the delay to active ingredient release from pharmaceutical dosage forms according to the invention. The core, which contains the active ingredient and releases it preferably in delayed manner, with an active ingredient content preferably of between 0.5 and 85 wt. %, particularly preferably of between 3.0 and 70 wt. % and very particularly preferably of between 8.0 and 66 wt. %, may be covered with additional active ingredient, which is released in undelayed manner as an initial dose, by various methods known to a person skilled in the art, for example pan coating, spraying of solutions or suspensions or by powder application methods, without this being absolutely essential for the desired delayed release simultaneously accompanied by rapid loading of the active ingredient for rapid pain relief on first administration of the pharmaceutical formulation according to the invention.

Further embodiments of the dosage form according to the invention are multilayer and jacketed tablets in which the active ingredient, in one or more layers of the multilayer tablet with an active ingredient content of preferably between 0.5 and 85 wt. %, particularly preferably between 3.0 and 70 wt. % and very particularly preferably between 8.0 and 66 wt. % or in the core of the jacketed tablet with an active ingredient content of preferably between 0.5 and 85 wt. %, particularly preferably between 3.0 and 70 wt. % and very particularly preferably between 8.0 and 66 wt. % is released in controlled manner by a polymer matrix and release of the active ingredient in one or more layers of the multilayer tablet or the outer jacket layer of the jacketed tablets proceeds in undelayed manner. Multilayer and jacketed tablets may contain one or more coatings which contain no active ingredient.

The dosage forms according to the invention may, for example, be produced by the following general method:

The constituents of the dosage form (active ingredient, polymers for forming the polymer matrix [matrix formers] and optional constituents) are weighed out in succession and then screened on a conventional screening machine. The Quadro Comil U10 screening machine may be used for this purpose, for example, a normal screen size being approx. 0.813 mm. The screened composition is then mixed in a container mixer, for example in a Bohle container mixer; typical operating conditions are: duration approx. 15 min±45 s at a rotational speed of 20±1 rpm. The resultant powder mixture is then pressed in a tabletting press to form a tablet. A Korsch EK0 tabletting press with a 10 mm diameter round, biconvex punch may for example be used for this purpose. Alternatively, the powder mixture may also be compacted and the compression mouldings subsequently screened (Comil 3 mm abrasive cutting screen followed by 1.2 mm round hole screen), the resultant granular product then being pressed as described above with the addition of lubricant (for example magnesium stearate), for example on an EK0 tabletting press with 10 mm round punches. Granulation may also be performed by wet granulation based on aqueous or organic solvents; aqueous solvents with or without suitable binders are preferred. The production method can straightforwardly be adapted to particular requirements and the desired dosage form in accordance with methods well known in the prior art.

The production of pharmaceutical dosage forms according to the invention is characterised by elevated reproducibility of the release characteristics of the compositions obtained, which contain 3-(2-dimethylaminomethylcyclohexyl)phenol or a pharmaceutically acceptable salt thereof. The release profile of the dosage forms according to the invention has proven to be stable over a period of storage of at least one year under conventional storage conditions in accordance with the ICH Q1AR Stability Testing Guideline.

A further aspect of the invention relates to a pharmaceutical composition comprising the active ingredient 3-(2-dimethylaminomethylcyclohexyl)phenol or one of the pharmaceutically acceptable salts thereof, preferably (1R,2R)-3-(2-dimethylaminomethylcyclohexyl)phenol, as such and/or as a pharmaceutically acceptable salt, and a cellulose ether or cellulose ester which, at a concentration of 2.0 wt. % in an aqueous solution at 20° C. has a viscosity in the range from 3,000 to 150,000 mPa·s.

The composition according to the invention is suitable for producing the dosage form according to the invention.

In a preferred embodiment of the composition according to the invention, the cellulose ether or cellulose ester is selected from the group consisting of methylcellulose, ethylcellulose, hydroxyethylcellulose, hydroxypropylcellulose, carboxymethylcellulose and hydroxypropylmethylcellulose.

Further preferred embodiments of the cellulose ether or cellulose ester are listed above in connection with the explanation of the dosage form according to the invention and also apply correspondingly for the composition according to the invention.

The present invention also provides the use of 3-(2-dimethylaminomethylcyclohexyl)phenol or one of the pharmaceutically acceptable salts thereof for producing an above-described dosage form or an above-described composition for combatting pain. The pain is preferably selected from the group consisting of acute pain and chronic pain, in particular inflammatory pain or neuropathic pain, wherein the pain may be weak, moderately severe, severe or extreme.

The combatting of pain is preferably accompanied by a significant reduction in the side-effect nausea and/or vomiting in comparison with a dosage form without controlled release. Administration preferably proceeds orally.

The present invention also provides the use of the active ingredient 3-(2-dimethylaminomethylcyclohexyl)phenol or one of the pharmaceutically acceptable salts thereof for producing a dosage form with controlled active ingredient release for combatting pain with a significant reduction in the side-effect nausea and/or vomiting in comparison with a dosage form without controlled release. Administration preferably proceeds orally. The pain is preferably selected from acute pain and chronic pain.

The present invention also provides a method for combatting pain comprising the administration of a pharmaceutically active quantity of the active ingredient 3-(2-dimethylaminomethylcyclohexyl)phenol or one of the pharmaceutically acceptable salts thereof to a patient, wherein the peak plasma level of the active ingredient is reached after 2 to 10 h. Administration preferably proceeds orally. The pain is preferably selected from acute pain and chronic pain.

The present invention also provides a method for combatting pain comprising the administration of a dosage form with controlled release containing a pharmaceutically active quantity of the active ingredient 3-(2-dimethylaminomethylcyclohexyl)phenol or one of the pharmaceutically acceptable salts thereof to a patient with a significant reduction in the side-effect nausea and/or vomiting in comparison with a dosage form without controlled release. Administration preferably proceeds orally. The pain is preferably selected from acute pain and chronic pain.

The following Examples serve to illustrate the present invention and preferred exemplary embodiments, but should not be interpreted as limiting.

### EXAMPLE 1

Matrix tablets with the following composition per tablet

| | mg | wt. % |
|---|---|---|
| (1R,2R)-3-(2-Dimethylaminomethylcyclohexyl)phenol | 60 | 25 |
| Hydroxypropylmethylcellulose, 100,000 mPa · s | 100 | 41.7 |
| Microcrystalline cellulose (Avicel PH 102 from FMC) | 77.5 | 32.3 |
| Highly disperse silicon dioxide | 1.25 | 0.5 |
| Magnesium stearate | 1.25 | 0.5 |
| Total quantity | 240 | 100 |

were produced in the following manner in a batch size of 1000 tablets:

All the constituents were weighed out and screened in a Quadro Comil U10 screening machine using a screen size of 0.813 mm, mixed in a container mixer (Bohle LM 40) for 15 min±15 s at a rotational speed of 20±1 rpm and pressed on a Korsch EK0 eccentric press to form biconvex tablets with a diameter of 10 mm, a radius of curvature of 8 mm and an average tablet weight of 240 mg.

In vitro release was determined using the Ph.Eur. paddle method at 75 rpm in 900 ml of pH 6.8 buffer to Ph.Eur. at 37° C. and with detection by UV spectrometry and is stated in the following table.

| Time | Total quantity of active |
|---|---|
| [min] | ingredient released [%] |
| 0 | 0 |
| 30 | 22 |
| 60 | 33 |
| 120 | 47 |
| 180 | 57 |
| 240 | 65 |
| 360 | 77 |
| 480 | 85 |
| 600 | 91 |
| 720 | 95 |

### EXAMPLE 2

Matrix tablets with the following composition per tablet

| | A | | B | | C | | D | |
|---|---|---|---|---|---|---|---|---|
| | mg | wt. % | mg | wt. % | mg | wt. % | mg | wt. % |
| (1R,2R)-3-(2-Dimethylamino- | 15 | 6 | 40 | 16 | 80 | 32 | 100 | 40 |
| methylcyclohexyl)phenol | | | | | | | | |
| Hydroxypropylmethylcellulose, | 70 | 28 | 70 | 28 | 70 | 28 | 70 | 28 |
| 100.000 mPa · s | | | | | | | | |
| Microcrystalline cellulose | 162.5 | 65 | 137.5 | 55 | 97.5 | 39 | 77.5 | 31 |
| Highly disperse silicon dioxide | 1.25 | 0.5 | 1.25 | 0.5 | 1.25 | 0.5 | 1.25 | 0.5 |
| Magnesium stearate | 1.25 | 0.5 | 1.25 | 0.5 | 1.25 | 0.5 | 1.25 | 0.5 |
| Total quantity | 250 | 100 | 250 | 100 | 250 | 100 | 250 | 100 |

were produced in a manner similar to the method stated in Example 1.

In vitro release was determined as in Example 1.

| | |
|---|---|
| Time | Total quantity of active ingredient released [%] |

| [min] | A | B | C | D |
|---|---|---|---|---|
| 0 | 0 | 0 | 0 | 0 |
| 30 | 27 | 23 | 20 | 19 |
| 60 | 37 | 33 | 32 | 31 |
| 120 | 51 | 48 | 48 | 47 |
| 180 | 60 | 58 | 60 | 59 |
| 240 | 68 | 68 | 69 | 69 |
| 360 | 81 | 81 | 82 | 83 |
| 480 | 90 | 89 | 90 | 92 |
| 600 | 96 | 95 | 95 | 96 |
| 720 | 100 | 100 | 98 | 99 |

### EXAMPLE 3

Matrix tablets with the following composition per tablet

| A | B | C | mg | wt. % |
|---|---|---|---|---|
| (1R,2R)-3-(2- | (1R,2R)-3-(2- | (1R,2R)-3-(2- | 80 | 32 |
| Dimethylaminomethyl- | Dimethylaminomethyl- | Dimethylamino- | | |
| cyclohexyl)phenol | cyclohexyl)phenol | methylcyclo- | | |
| | | hexyl)phenol | | |
| Hydroxypropylmethyl- | Hydroxypropylmethyl- | Hydroxypropylmethyl- | 70 | 28 |
| cellulose, | cellulose, | cellulose, | | |
| 100,000 mPa · s | 100,000 mPa · s | 100,000 mPa · s | | |
| Microcrystalline | Calcium | Lactose | 97.5 | 39 |
| cellulose | hydrogenphosphate | monohydrate | | |
| Highly disperse silicon | Highly disperse silicon | Highly disperse silicon | 1.25 | 0.5 |
| dioxide | dioxide | dioxide | | |
| Magnesium stearate | Magnesium stearate | Magnesium stearate | 1.25 | 0.5 |
| Total quantity | Total quantity | Total quantity | 250 | 100 |

were produced in a batch size of 75 tablets in a manner similar to the method stated in Example 1.

In vitro release was determined as in Example 1.

| Time | Total quantity of active ingredient released [%] | | |
|---|---|---|---|
| [min] | A | B | C |
| 0 | 0 | 0 | 0 |
| 30 | 19 | 21 | 21 |
| 60 | 31 | 32 | 33 |
| 120 | 47 | 48 | 49 |
| 180 | 58 | 59 | 59 |
| 240 | 67 | 67 | 68 |
| 360 | 78 | 78 | 78 |
| 480 | 85 | 84 | 84 |
| 600 | 89 | 88 | 88 |
| 720 | 92 | 90 | 90 |

### EXAMPLE 4

Matrix tablets with the following composition per tablet

| | mg | wt. % |
|---|---|---|
| (1R,2R)-3-(2-Dimethylaminomethylcyclohexyl)phenol | 40 | 16 |
| Hydroxypropylmethylcellulose, 100,000 mPa · s | 70 | 28 |
| Microcrystalline cellulose | 137.5 | 55 |
| Highly disperse silicon dioxide | 1.25 | 0.5 |
| Magnesium stearate | 1.25 | 0.5 |
| Total quantity | 250 | 100 |

were produced in a batch size of 100 tablets in a manner similar to the method stated in Example 1.

In vitro release was determined under the following conditions:
(A): as described in Example 1;
(B): application of Ph.Eur. paddle method at 75 rpm in 900 ml pH 1.2 buffer to USP 22 at 37° C. and with detection by UV spectrometry;
(C): application of Ph.Eur. paddle method at 75 rpm, a pH of 1.2 being established from 0-30 min, a pH of 2.3 from 30-120 min, a pH of 6.5 from 120-180 min and a pH of 7.2 for the remainder of the test period.

The table states the results for the various test conditions:

| Time | Total quantity of active ingredient released [%] | | |
|---|---|---|---|
| [min] | A | B | C |
| 0 | 0 | 0 | 0 |
| 30 | 19 | 18 | 18 |
| 60 | 32 | 31 | 32 |
| 120 | 48 | 46 | 47 |
| 180 | 60 | 59 | 60 |
| 240 | 68 | 67 | 67 |
| 360 | 79 | 78 | 77 |
| 480 | 87 | 86 | 84 |
| 600 | 92 | 92 | 90 |
| 720 | 95 | 95 | 94 |

### EXAMPLE 5

Matrix tablets with the following composition per tablet

| | mg | wt. % |
|---|---|---|
| (1R,2R)-3-(2-Dimethylaminomethylcyclohexyl)phenol | 80 | 32 |
| Hydroxypropylmethylcellulose 100,000 mPa · s | 70 | 28 |
| Microcrystalline cellulose | 97.5 | 39 |
| Highly disperse silicon dioxide | 1.25 | 0.5 |
| Magnesium stearate | 1.25 | 0.5 |
| Total quantity | 250 | 100 |

were produced in a batch size of 100 tablets in a manner similar to the method stated in Example 1. Press-moulding was carried out with different pressing forces, such that tablets with the following breaking strengths were obtained:

### (A) 60 N, (B) 80 N, (C) 100 N, (D) 150 N.

In vitro release was determined as in Example 1.

| | |
|---|---|
| Time | Total quantity of active ingredient released [%] |

| [min] | A | B | C | D |
|---|---|---|---|---|
| 0 | 0 | 0 | 0 | 0 |
| 30 | 18 | 18 | 20 | 20 |
| 60 | 31 | 31 | 32 | 30 |
| 120 | 49 | 48 | 49 | 45 |
| 180 | 61 | 60 | 61 | 56 |
| 240 | 69 | 68 | 69 | 64 |
| 360 | 80 | 80 | 81 | 77 |
| 480 | 87 | 87 | 88 | 85 |
| 600 | 91 | 91 | 92 | 90 |
| 720 | 93 | 94 | 94 | 93 |

### EXAMPLE 6

Matrix tablets with the following composition per tablet

| A | B | C | mg | wt. % |
|---|---|---|---|---|
| (1R,2R)-3-(2- | (1R,2R)-3-(2- | (1R,2R)-3-(2- | 40 | 16 |
| Dimethylaminomethyl- | Dimethylaminomethyl- | Dimethylaminomethyl- | | |
| cyclohexyl)phenol | cyclohexyl)phenol | cyclohexyl)phenol | | |
| Hydroxypropylmethyl- | Hydroxypropylmethyl- | Hydroxypropylmethyl- | 70 | 28 |
| cellulose, 90SH, | cellulose, 60SH, | cellulose, 65SH, | | |
| 15,000 mPa · s | 4,000 mPa · s | 4,000 mPa · s | | |
| Microcrystalline | Calcium | Lactose monohydrate | 137.5 | 55 |
| cellulose | hydrogenphosphate | | | |
| Highly disperse silicon | Highly disperse silicon | Highly disperse silicon | 1.25 | 0.5 |
| dioxide | dioxide | dioxide | | |
| Magnesium stearate | Magnesium stearate | Magnesium stearate | 1.25 | 0.5 |
| Total quantity | Total quantity | Total quantity | 250 | 100 |

were produced in a batch size of 200 tablets in a manner similar to the method stated in Example 1.

In vitro release was determined as in Example 1.

| Time | Total quantity of active ingredient released [%] | | |
|---|---|---|---|
| [min] | A | B | C |
| 0 | 0 | 0 | 0 |
| 30 | 19 | 22 | 21 |
| 60 | 34 | 35 | 33 |
| 120 | 52 | 51 | 47 |
| 180 | 65 | 62 | 57 |
| 240 | 73 | 69 | 65 |
| 360 | 83 | 79 | 75 |
| 480 | 90 | 84 | 82 |
| 600 | 92 | 88 | 86 |
| 720 | 94 | 90 | 89 |

### EXAMPLE 7

Matrix tablets with the following composition per tablet

| [mg] | mg | wt. % |
|---|---|---|
| (1R,2R)-3-(2-Dimethylamino- | 209.64 | 38.12 |
| methylcyclohexyl)phenol | | |
| Hypromellose 15,000 mPa · s | 60.00 | 10.91 |
| Lactose, type 200 | 268.36 | 48.79 |
| Colloidal anhydrous silicon | 6.00 | 1.09 |
| dioxide | | |
| Magnesium stearate | 6.00 | 1.09 |
| Weight of tablet core | 550.00 | 100.00 |

were produced in a batch size of 1,000 tablets. To this end, all the tablet constituents were weighed out, screened through a 0.315 mm screen, mixed, granulated with water in a Kenwood mixer, pressed through a 1 mm screen, dried at 50° C. in a Hoirden drying cabinet and press-moulded on a Korsch EK0 eccentric tablet press with 7×17 mm oblong punches [to form tablets] weighing 550 mg per tablet.

The tablets exhibited the following in vitro release properties:

| Time | Total quantity of active |
|---|---|
| [min] | ingredient released [%] |
| 0 | 0 |
| 30 | 16 |
| 240 | 61 |
| 480 | 92 |

### EXAMPLE 8

a) Film coated tablets having the following composition per tablet

| [mg] | PR (A) | PR (B) | PR (C) |
|---|---|---|---|
| (1R,2R)-3-(2-Dimethylamino- | 60.00 | 60.00 | 60.00 |
| methylcyclohexyl)phenol | | | |
| Hypromellose 100,000 mPa · s | 50.00 | 100.00 | 200.00 |
| (Shin-Etsu) | | | |
| Microcrystalline cellulose | 137.50 | 87.50 | 85.00 |
| (Avicel 102, FMC) | | | |
| Highly disperse silicon dioxide | 1.25 | 1.25 | 2.50 |
| Magnesium stearate | 1.25 | 1.25 | 2.50 |
| Weight of tablet core | 250.00 | 250.00 | 350.00 |

were produced in a batch size of 1,000 tablets. To this end, all the tablet core constituents were weighed out, screened through a 0.315 mm screen, mixed and press-moulded on a Fette P1200 rotary tablet press with 10 mm punches and a radius of curvature of 8 mm. The tablets (formulation A and B) were then film-coated with an aqueous lacquer suspension (approx. 29% solids content) prepared from hypromellose 6 mPa·s, Macrogol 6000, propylene glycol, talcum, titanium dioxide and purified water, until the tablet weight had risen by 12 mg.

The film coated tablets exhibited the following in vitro release values:

| Time | Total quantity of active ingredient released [%] | | |
|---|---|---|---|
| [min] | PR (A) | PR (B) | PR (C) |
| 0 | 0 | 0 | 0 |
| 30 | 22 | 18 | 14 |
| 180 | 65 | | |
| 240 | | 62 | 47 |
| 360 | 85 | | |
| 480 | | 84 | |
| 720 | | | 80 |

As the above table shows, 80% of the active ingredient in formulations PR (A), PR (B) and PR (C) were released in vitro after approx. 360 min, 480 min and 720 min respectively.

b) In an open, randomised phase 1 four-way crossover study, the pharmacokinetic parameters for these three dosage forms according to the invention exhibiting different release behaviour [prolonged release, PR (A), PR (B) and PR (C)] were determined in vivo and compared with an active ingredient solution [immediate release, IR] as a reference formulation. 1 ml of the IR active ingredient solution contained:

| | | |
|---|---|---|
| 10.0 | mg | (1R,2R)-3-(2-dimethylaminomethylcyclohexyl)phenol |
| 7.0 | mg | sodium chloride, Ph. Eur., for parenteral use |
| 0.5 | mg | sodium citrate · 2H₂O, Ph. Eur., for parenteral use |
| 985.5 | mg | water for injection, Ph. Eur.* |

Production proceeded in accordance with the standard procedure for solutions for injection; the solution was packaged in 1 ml portions in ampoules.

A dose D of 60 mg of (1R,2R)-3-(2-dimethylaminomethylcyclohexyl)phenol was administered to each of 8 volunteer female test subjects and the plasma concentration of the active ingredient was measured over 32 hours.

Quantitative analysis of the concentration of (1R,2R)-3-(2-dimethylaminomethylcyclohexyl)phenol in the blood plasma was carried out using O-desmethyltramadol (M1) as internal standard. The active ingredients were extracted from the samples by liquid-liquid extraction with tert.-butyl methyl ether. The extracts were analysed with HPLC by fluorometric detection. The calibration curves exhibited signal linearity in the plasma concentration range from 0.23 ng/ml to 92 ng/ml.

The results are shown in FIGS. 1A and 1B. FIG. 1A shows a linear representation of plasma concentration (y-axis), FIG. 1B a logarithmic representation.

Pharmacokinetic parameters were calculated by noncompartmentalised analysis using the validated software package MODUNA, which was developed by Grunenthal GmbH.

The calculations were carried out using all available decimal places without rounding.

The first subarea from administration until the first valid plasma concentration was calculated, depending on the type of administration, by extrapolating a concentration value C_{tdose} to the time of administration tdose. If t_{lag} was >0 h, the first subarea was calculated from t_{lag}.

AUC was calculated by summation from the subarea AUC₀₋ₜ (area under the concentration/time curve with measured concentrations above the detection limit) and the residual area AUC_{t-∞} (=ĉₜ/λ_{z}), in which ĉₜ is the plasma concentration estimated on the basis of a logarithmic linear regression at the time of the final measurement at which a plasma concentration which was still above the detection limit was determined.

AUC_{0-∞}T was calculated by numerical integration using the trapezium rule (Gibaldi and Perrier, 1982). The linear trapezium rule was applied up to Cₘₐₓ and thereafter the logarithmic trapezium rule. If two or more plasma concentration maxima were observed, Cₘₐₓ was determined for the first maximum.

The rate constant of the terminal elimination phase λ_{z} was determined by logarithmic linear regression of the terminal phase of the plasma concentration curves (Cawello, 1999). The λ_{z} time interval [h] for the regression was defined according to the following table:

| | Female test subject | | | | | | | |
|---|---|---|---|---|---|---|---|---|
| | 1 | 2 | 3 | 4 | 5 | 6 | 7 | 8 |
| PR(A) | 10-32 | 10-32 | 10-32 | 10-32 | 10-32 | 10-32 | 10-32 | 10-32 |
| PR(B) | 10-32 | 10-32 | 10-32 | 10-32 | 10-32 | 10-32 | 10-32 | 10-32 |
| PR(C) | 16-32 | 10-28 | 10-32 | 10-32 | 10-32 | 13-32 | 13-32 | 10-32 |
| IR | 20-32 | 10-32 | 10-32 | 10-32 | 10-32 | 10-32 | 20-32 | 13-28 |

When determining the half value duration HVD, the time at which the plasma concentration was 50% of Cₘₐₓ was determined by linear interpolation.

The following table summarises the pharmacokinetic parameters calculated from the measured plasma concentrations:

| | PR (A) | | PR (B) | | PR (C) | | IR [Comparison] | |
|---|---|---|---|---|---|---|---|---|
| D = 60 mg | Mean | CV [%] | Mean | CV [%] | Mean | CV [%] | Mean | CV [%] |
| tₘₐₓ [h] | 5.00 | 18.5 | 4.50 | 23.8 | 4.75 | 27.0 | 1.25 | 37.0 |
| t_{1/2, z} [h] | 6.68 | 23.5 | 7.44 | 27.7 | 12.10 | 32.5 | 5.69 | 25.0 |
| MRT [h] | 11.75 | 11.1 | 13.47 | 19.1 | 19.59 | 28.5 | 7.28 | 16.7 |
| HVD [h] | 9.40 | 23.1 | 12.05 | 24.5 | 15.07 | 39.0 | 4.78 | 42.8 |
| λ_{z} [h⁻¹] | 0.109 | 23.4 | 0.099 | 26.2 | 0.063 | 33.9 | 0.128 | 22.2 |
| V(z)/f [l] | 1,569 | 40.8 | 1,665 | 29.5 | 2,817 | 52.7 | 1,102 | 26.1 |
| CL/f [ml min⁻¹] | 2,708 | 34.9 | 2,747 | 44.0 | 2,800 | 54.7 | 2,392 | 43.7 |
| AUC₀₋ₜ [h ng ml⁻¹] | 343 | 39.3 | 341 | 41.7 | 310 | 42.9 | 404 | 32.5 |
| AUC [h ng m⁻¹] | 359 | 38.6 | 369 | 41.4 | 384 | 44.3 | 411 | 33.0 |
| Cₘₐₓ [ng ml⁻¹] | 32.2 | 58.3 | 26.8 | 65.6 | 19.7 | 38.0 | 64.2 | 40.9 |
| Cₘₐₓ/D [l⁻¹] | 5.37 10⁻⁴ | — | 4.48 10⁻⁴ | — | 3.28 10⁻⁴ | — | 1.07 10⁻³ | — |
| Cₘₐₓ/AUC [h⁻¹] | 0.090 | — | 0.073 | — | 0.051 | — | 0.156 | — |

The controlled-release formulations (prolonged release PR (A), PR (B) and PR (C)) which under in vitro conditions release 80% of the active ingredient after approx. 360 min, 480 min and 720 min respectively, also exhibited an increasing delayed-release action in vivo in humans.

While the average value for Cₘₐₓ on administration of the active ingredient solution was 64.2 ng/ml (comparison, immediate release, IR), it declined to 32.2 ng/ml (PR(A)), 26.8 ng/ml (PR(B)) and 19.7 ng/ml (PR(C)) on administration of the formulations according to the invention.

The average value for tₘₐₓ was similar for all three formulations according to the invention and was in the range from 4.5 to 5.0 h, but was clearly delayed in comparison with administration of the active ingredient solution (1.25 h).

The average half value duration HVD increased from 4.78 h on administration of the active ingredient solution to 9.40 h (PR (A)), 12.05 (PR (B)) and 15.07 h (PR (C)). The results for the mean residence time confirmed this trend with increases from 7.28 h on administration of the active ingredient solution to up 19.59 h (PR (C)).

The half-life during the terminal elimination phase increased from 5.69 h on administration of the active ingredient solution to 6.68 h (PR (A)), 7.44 (PR (B)) and 12.10 h (PR (C)). An adequate delayed-release action in comparison with a immediate release formulation (active ingredient solution, succus, IR) is characterised by halving Cₘₐₓ or doubling HVD while simultaneously increasing t_{1/2,z}, ideally without in so doing changing the AUC.

In one study, the PTF % individual values after twice daily administration were between 49% and 88% in a dose range from 160 to 400 mg daily dose. The average values within the dose groups were between 57% and 64%.

c) It has been found in clinical testing that, in comparison with conventional dosage forms intended for oral administration of 3-(2-dimethylaminomethylcyclohexyl)phenol, it is possible to achieve a significant reduction in side-effects, in particular nausea and/or vomiting.

This has inter alia the advantage that the therapeutic range (ratio of the therapeutic dose to the toxic active ingredient dose) of 3-(2-dimethylaminomethylcyclohexyl)phenol is increased.

1-33. (canceled)

34. A dosage form for controlled release of the active ingredient 3-(2-dimethylaminomethylcyclohexyl)phenol or a pharmaceutically acceptable salt thereof, wherein said dosage form achieves in vivo a peak plasma level of said active ingredient after 2 to 10 hours.

35. A dosage form for controlled release of the active ingredient 3-(2-dimethylaminomethylcyclohexyl)phenol or a pharmaceutically acceptable salt thereof,
wherein said dosage form releases in vitro,
after 0.5 hour 3.0 to 37 wt. %,
after 1 hour 5.0 to 56 wt. %,
after 2 hours 10 to 77 wt. %,
after 3 hours 15 to 88 wt. %,
after 6 hours at least 30 wt. %,
after 12 hours at least 50 wt. %,
after 18 hours at least 70 wt. %, and
after 24 hours at least 80 wt. %
of said active ingredient originally contained in the dosage form; and
wherein the release of said active ingredient is measured in accordance with the European Pharmacopoeia with a paddle stirrer apparatus in buffer at a pH value of 6.8, a temperature of 37° C. and 75 rpm.

36. A dosage form as claimed in claim 34, wherein the release of said active ingredient from said dosage form satisfies the relation:
0.010 hour⁻¹≦Cₘₐₓ/AUC≦0.150 hour⁻¹.,
wherein Cₘₐₓ represents the maximum measured plasma concentration of the active ingredient, and AUC represents the area under the plasma concentration/time curve.

37. A dosage form as claimed in claim 34, wherein at an identical dose D, t_{1/2,z} is higher than in a comparison formulation without controlled release.

38. A dosage form as claimed in claim 34, wherein t_{1/2,z}>5.7 hours.

39. A dosage form as claimed in claim 34, wherein said dosage form produces a mean residence time greater than 7.5 hours.

40. A dosage form as claimed in claim 34, wherein said dosage form produces a half value duration greater than 5.0 hours.

41. A dosage form as claimed in claim 34, wherein said dosage form contains an active ingredient dose D, and upon administration of said dosage form, the release of said active ingredient from said dosage form satisfies the relation:
7.0 10⁻⁵ l⁻¹≦Cₘₐₓ/D≦1.05 10⁻³ l⁻¹,
where Cₘₐₓ represents a maximum measured plasma concentration.

42. A dosage form as claimed in claim 34, wherein upon twice daily administration, said dosage form produces a peak to trough fluctuation of less than 80%.

43. A dosage form as claimed in claim 34, wherein said dosage form comprises a polymer matrix from which at least a portion of the total dose of said active ingredient contained in the dosage form is released in a delayed manner.

44. A dosage from as claimed in claim 34, wherein said dosage form comprises a film coating which releases at least a portion of the total dose of said active ingredient contained in the dosage form in a delayed manner.

45. A dosage form as claimed in claim 34, wherein said dosage form comprises a polymer matrix in which at least a portion of said active ingredient is embedded, said polymer matrix being based on a cellulose ether or cellulose ester which in an aqueous solution at a concentration of 2.0 wt. % at 20° C. has a viscosity in the range from 3,000 to 150,000 mPa·s.

46. A dosage form as claimed in claim 45, wherein the cellulose ether or cellulose ester is selected from the group consisting of methylcellulose, ethylcellulose, hydroxyethylcellulose, hydroxypropylcellulose, carboxymethylcellulose and hydroxypropylmethylcellulose.

47. A dosage form as clamed in claim 45, wherein polymer matrix comprises from 5.0 to 85 wt.-% of the total weight of the dosage form.

48. A dosage form as claimed in claim 45, wherein the relative weight ratio of the polymer matrix to the active ingredient is in the range from 3:1 to 1:10.

49. A dosage form as claimed in claim 45, wherein the polymer matrix comprises from 15 to 35 wt. % of a hydroxypropylmethylcellulose relative to the total weight of the dosage form; said hydroxypropylmethylcellulose having a viscosity in the range from 50,000 to 130,000 mPa·s in an aqueous solution at a concentration of 2.0 wt. % and at 20° C.

50. A dosage form as claimed in claim 45, wherein said dosage form contains a filler in an amount such that the relative weight ratio of the filler to the polymer matrix is less than 6:1.

51. A dosage form as claimed in claim 50, wherein said filler is selected from the group consisting of:
fillers soluble in an aqueous medium;
non-swelling fillers insoluble in an aqueous medium, and
swelling fillers insoluble in an aqueous medium.

52. A dosage form as claimed in claim 34, wherein the active ingredient is (1R,2R)-3-(2-dimethylaminomethylcyclohexyl)phenol or a pharmaceutically acceptable salt thereof.

53. A dosage form as claimed in claim 34, wherein said dosage form comprises from 0.5 to 85 wt.-% of said active ingredient relative to the total weight of the dosage form.

54. A dosage form as claimed in claim 34, wherein said dosage form produces an in vivo peak plasma level of said active ingredient from 3 to 8 hours after administration.

55. A dosage form as claimed in claim 34, wherein said dosage form is formulated for once or twice daily administration.

56. A dosage form as claimed in claim 34, wherein said dosage form is formulated for oral or rectal administration.

57. A dosage form as claimed in claim 34, wherein said dosage form is in tablet form.

58. A pharmaceutical composition comprising:
3-(2-dimethylaminomethylcyclohexyl)phenol or a pharmaceutically acceptable salt thereof, and
a cellulose ether or cellulose ester which in an aqueous solution at a concentration of 2.0 wt. % and at 20° C. has a viscosity in the range from 3,000 to 150,000 mPa·s.

59. A composition as claimed in claim 58, wherein said cellulose ether or cellulose ester is selected from the group consisting of methylcellulose, ethylcellulose, hydroxyethylcellulose, hydroxypropylcellulose, carboxymethylcellulose and hydroxypropylmethylcellulose.

60. A method of treating pain in a subject, said method comprising administering to said subject an effective pain treating amount of 3-(2-dimethylaminomethylcyclohexyl)phenol or a pharmaceutically acceptable salt thereof in a pharmaceutical dosage form as claimed in claim 34, whereby the treatment is accompanied by a reduction in side-effect nausea or vomiting or both in comparison to treatment with 3-(2-dimethylaminomethylcyclohexyl)phenol or a pharmaceutically acceptable salt thereof in a dosage form without controlled release.

61. A method as claimed in claim 60, wherein the pharmaceutical dosage form is administered orally.

62. A method as claimed in claim 60, wherein said pain is acute pain or chronic pain.

63. A method of treating pain in a subject, said method comprising administering to said subject an effective pain treating amount of 3-(2-dimethylaminomethylcyclohexyl)phenol or a pharmaceutically acceptable salt thereof in a pharmaceutical dosage form as claimed in claim 34, wherein said pharmaceutical dosage form reaches a peak plasma level of said active ingredient from 2 to 10 hours after administration.
